(19) United States
(12) Patent Application Publication
(10) Pub. No.: US 2008/0319055 A1
(43) Pub. Date: Dec. 25, 2008

(54) Cooling Compounds

(75) Inventors: Lucienne Cole, Cincinnati, OH (US); Stefan Michael Furrer, Cincinnati, OH (US); Christophe C. Galopin, Chesterfield, VA (US); Pablo Victor Krawec, Cincinnati, OH (US); Jay Patrick Slack, Loveland, OH (US)

Correspondence Address: CURATOLO SIDOTI CO., LPA, 24500 CENTER RIDGE ROAD, SUITE 280, CLEVELAND, OH, 44145, US

(73) Assignee: GIVAUDAN SA, Vernier (CH)

(21) Appl. No.: 11/884,980

(22) PCT Filed: Mar. 15, 2006
(86) PCT No.: PCT/CH06/00150

§ 371 Date: Nov. 14, 2007

Related U.S. Application Data
(60) Provisional application No. 60/664,804, filed on Mar. 24, 2005.

(51) Int. Cl. A61K 31/166; A61K 31/277; A61K 31/235; A61K 31/357; C07C 229/54; C07C 255/58; C07C 233/67; C07D 317/68

(52) U.S. Cl. 514/464; 514/617; 514/619; 514/538; 560/41; 558/415; 564/183; 549/436

(57) ABSTRACT

A method of providing a cooling sensation to the skin or the mucous membranes of the body by applying thereto a compound of Formula I

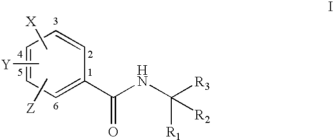

in which X is H or (CH₂)ₙ—R, n is 0 or 1, Y and Z are selected independently from the group consisting of H, OH, phenyl, C1-C4 straight or branched alkyl, or, a C₁-C₄ straight or branched alkoxy, or X and Y form together a bivalent radical selected from the group consisting of —O—CH₂—O—, —N═CH—O— and —N═CH—S— which forms together with the carbon atoms to which they are attached a 5-membered ring; and
R is a group with non-bonding electrons, R¹ is H or C₁-C₅ branched alkyl, R2 and R3 are C₁-C4 branched alkyls, or R² and R³ taken together form a monocyclic, bicyclic or tricyclic radical of up to 10 carbons provided that R¹, R² and R³ together comprise at least 6 carbons.

The compounds may be incorporated into products such as dentifrices, foodstuffs, confectionery, beverages, and cosmetic and medicinal preparations.

This invention relates to a method for providing a cooling sensation and to compounds that provide this effect.

Cooling compounds, that is, chemical compounds that impart a cooling sensation to the skin or the mucous membranes of the body, are well known to the art and are widely used in a variety of products such as foodstuffs, tobacco products, beverages, dentifrices, mouthwashes and toiletries.

One class of cooling compounds that has enjoyed substantial success is that of the N-substituted p-menthane carboxamides. Examples of these compounds are described in, for example, British Patent GB 1,421,744.

Although some of the compounds of the prior art have been successfully commercialized, the intricate structures of the carboxamide moiety make difficult to produce. These chemicals can only be offered at high price, which limits their use in consumer products.

It has now been found that a category of simple arylcarboxamides of formula I are good cooling compounds and can easily be made from commercially available benzoic acids. The invention therefore provides a method of providing a cooling sensation to the skin or the mucous membranes of the body by applying thereto a compound of Formula I

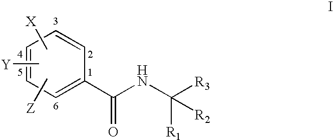

in which X is H or (CH₂)ₙ—R, n is 0 or 1, Y and Z are selected independently from the group consisting of H, OH, phenyl, C₁-C₄ straight or branched alkyl, or, a C₁-C₄ straight or branched alkoxy, or X and Y form together a bivalent radical selected from the group consisting of —O—CH₂—O—, —N═CH—O— and —N═CH—S— which forms together with the carbon atoms to which they are attached a 5-membered ring, i.e. a 1,3-dioxolane ring, a 1,3-oxazole ring or a 1,3-thiazole ring respectively; and
R is a group with non-bonding electrons, R¹ is H, CH₃, C₂H₅ or C₃-C₅ branched alkyl, R² and R³ are CH₃, C₂H₅ or C₃-C₄ branched alkyl, or two or more of R¹, R² and R³ taken together form a monocyclic, bicyclic or tricyclic radical of up to 10 carbons provided that R¹, R² and R³ together comprise at least 6 carbons.

R¹, R², R³ and the carbon to which they are attached may be, for example, para-menthyl, bornyl or adamantyl.

R¹, R², R³ may be chiral or racemic.

Particular compounds are those in which R¹ is methyl and R² and R³ are isopropyl, and in which R¹, R² and R³ are all ethyl.

Particular compounds are those in which X is in the 4-position. Other particular compounds are those in which X is in the 4-position and Y and Z are H, OH, OMe or methyl or X and Y form together a bivalent radical selected from the group consisting of —O—CH₂—O—, —N═CH—O— and —N═CH—S—, thus forming together with the carbon atoms to which they are attached a 5-membered ring, i.e. a 1,3-dioxolane ring, a 1,3-oxazole ring or a 1,3-thiazole ring respectively.

Particular groups R with non-bonding electrons are halogens, OH, OMe, NO₂, CN, Ac, SO₂NH₂, CHO, CO₂H, CONH₂, C1-C4 alkyl carboxylates such as CO₂Et, C1-C4 alkylamides such as CONHMe or heterocycles such as:

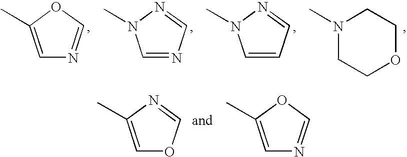

Particular R¹, R² and R3 combinations are:

| R¹ | R² | R³ |
|---|---|---|
| H | bornyl | |
| H | 3-p-menthyl | |
| H | isopropyl | isopropyl |
| methyl | isopropyl | isopropyl |
| ethyl | ethyl | ethyl |

A number of the compounds hereinabove defined are novel. Thus, in a further embodiment of the present invention, there is provided a compound of the formula I

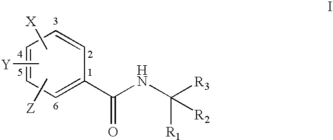

in which X, Y, Z, R¹, R², R³ are as hereinabove defined, and in which one of the following provisos applies:
(a) R¹ and X are not H, and, R¹, R², R³ and the carbon to which they are attached form an acyclic moiety;
(b) R¹ is H, and R², R³ and the carbon to which they are attached form an acyclic moiety, only one of R², R³ being isopropyl or tert-butyl;
(c) R¹ is H, R² and R³ are both isopropyl, and X is in the 4-position and is not H, halogen, Me, MeO, NO₂, aryl, methylenediaryl, N-(4-carbamimidoyl-phenyl)-6-methoxy-pyridine-2-carboxamide, N-(4-carbamimidoyl-phenyl)-benzamide, a heme derivative and R is not morpholine, N′-phenylpiperazine, phenylmercaptan, p-chlorophenylmercaptan, isoquinoline, an N-linked sulfonamide derivative or an aryl,
(d) R¹ is H and R² and R³ are both tert-butyl; X is not H;
(e) R¹ is H, R² and R³ together with the carbons to which they are attached form a p-menthane ring and X, Y and Z are not H;
(f) R¹ is H, R² and R³ together with the carbons to which they are attached form a p-menthane ring, Z is H and neither X or Y is H or OH;
(g) R¹ is H, R² and R³ together with the carbons to which they are attached form a p-menthane ring, Z is H, Y is OH and X is neither formamide nor NO₂;
(h) R¹ is H, R² and R³ together with the carbons to which they are attached form a p-menthane ring, Z and Y are both H, and X is not H, COOH, quinolinylsulfonamide, CF₃, a methylenediaryl or a heme derivative.

Particular examples of such compounds are those corresponding to Formulae II, III and IV, when provisos (a), (b) and (c) apply, and those corresponding to Formula V when provisos (e), (f), (g) and (h) apply:

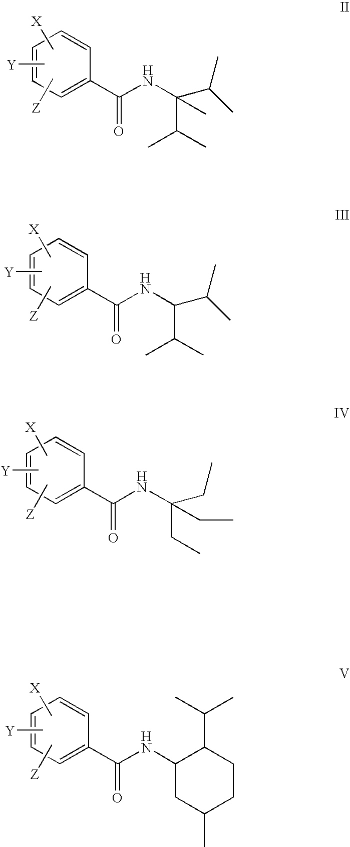

Particular examples of useful stereoisomers are (1R,2S,5R)-5-methyl-2-(1-methylethyl)-cyclohexanamine [(1R,2S,5R)-menthyl] and (2S,5R)-5-methyl-2-(1-methylethyl)-cyclohexanamine [(2S,5R)-menthyl].

The compounds may be easily prepared by amidation of a benzoyl chloride with an amine or an aminium chloride salt. Amines where R¹═H can be made from their corresponding ketone according to Schopohl, M. et al. Synthesis 2003, 17, 2689. Amines where R¹ is C1-C5 alkyl can be prepared from their corresponding alcohol according to Jirgensons, A et al. Synthesis 2000, 12, 1709-1712

The invention also provides a method of providing a cooling effect to a product that will be orally ingested, applied to the skin or used in a tobacco product, comprising the incorporation in the product of an effective amount of a compound as hereinabove defined. The invention further provides a composition that provides a cooling sensation to the skin or oral cavity, comprising an effective amount of a compound as hereinabove defined. The kinds of compositions in which the compounds hereinabove defined can be used include personal care products such as dentifrices (toothpastes, tooth gels, mouthwashes), cosmetic and medicinal preparations, such as tablets, lozenges, liquids, creams and sprays, foodstuffs and confectionery, hard candy, beverages, etc.

The “effective amount” required will naturally vary quite widely, depending on the natures of the compound and composition, the type of application and the extent and nature of cooling effect desired. As a result, any quantities given can only be approximations at best. However, typical concentrations are a maximum of 5000 ppm, that is, 0.5% by weight of the composition. As a general rule, between 50 and 3000 ppm are all that is required for a solid composition. In the case of beverages, as low as 15 ppm may be sufficient to generate a desired cooling effect.

In addition to the cooling compounds, the compositions may contain all the normal ingredients known to the art that are useful in such compositions, in art-recognised quantities.

More than one compound of the type hereinabove described may be used in the compositions according to this invention. In addition, the compounds may be used in conjunction with other known and/or commercially-available cooling compounds. Such compounds include menthol, menthone, isopulegol, N-ethyl p-menthanecarboxamide (WS-3), N,2,3-trimethyl-2-isopropylbutanamide (WS-23), menthyl lactate, menthone glycerine acetal (Frescolat® MGA), mono-menthyl succinate (Physcool®), mono-menthyl glutarate, O-menthyl glycerine (CoolAct® 10) and 2-sec-butylcyclohexanone (Freskomenthe®).

The incorporation of the compounds into the compositions may be achieved by entirely conventional means.

The invention is now further described with reference to the following non-limiting examples.

### EXAMPLE 1

### Preparation of Menthylamine

41.69 g of hydroxylamine hydrochloride were dissolved in 200 mL of water. Under ice cooling, 40 g of NaOH pellets were added. After the NaOH was dissolved, 61.7 g of L-Menthone were added over a period of 10 minutes. The mixture was stirred at RT for 70 h. The mixture, containing white solid balls, was extracted twice with MTBE. The organic layers were washed with water and brine, dried over MgSO₄ and concentrated to give 67 g of white solid which is reacted with 12 g of lithium aluminum hydride in 300 mL of MTBE, under ice cooling. The mixture was stirred for 96 hours at Room Temperature. The light grayish suspension was treated with acetone and with 40 mL of HCl (1N). The yellowish supernatant was acidified with HCl (37%) and extracted twice with MTBE. The organic layers were washed with HCl (1N). The combined aqueous layers were neutralized with NaOH pellets to pH 13 and extracted twice with MTBE. The organic layers were washed with brine, dried over MgSO₄ and concentrated to give 42.3 g of a yellowish liquid which is purified by distillation.

### EXAMPLE 2

### Preparation of 1-methyl-1-isopropylisobutylaminium chloride

18.1 g of 1-methyl-1-isopropylisobutanol and 15.74 g of Chloroacetonitrile are dissolved in 27.2 mL of acetic acid and the mixture was cooled in an ice bath. 27.3 g of sulfuric acid was added over a period of 20 minutes. The mixture was stirred at 0° C. for 1 h and for another 4 h at room temperature. The mixture was quenched with ice and extracted with MTBE. The organic layers were washed twice with NaHCO3, brine, dried over MgSO₄ and concentrated to recover 32.3 g of a yellow oil containing N-1-methyl-1-isopropylisobutyl 1-chloroacetamide with the following properties:

1HNMR (300 MHz, CDCl3) δ in ppm (two rotomers): 6.4 and 6.05 (broad s., 1H), 3.92 and 3.97 (d, 2H), 2.09 and 1.93 (m, 2H), 1.37 and 1.32 (d, 3H), 0.93 and 0.84 (m, 12H) 13CNMR (75 MHz, CDCl3) δ in ppm (two rotomers): 165, 164.5, 63.1, 58.2, 45.25, 43.4, 43.1, 34.35, 26.95, 26.9, 24.6, 24.2, 17.7, 17.5, 14.7, 16.6, 8.35

MS/EI: 207 (M^{+·}), 205 (M^{+·}), 192, 190, 164, 162, 150, 148, 136, 134, 97

This oil was mixed with 13.7 g of thiourea and 50 mL of acetic acid in 250 mL of ethanol. The mixture was heated at reflux overnight. 500 mL of water was added and the suspension was stirred at room temperature for 30 minutes. NaOH pellets are added to set the solution to alkaline pH. The yellowish solution was extracted three times with pentane and the organic layers were washed with brine and dried over MgSO₄. 1 L of HCl in Et2O (1M) was added and the mixture was stirred at room temperature for 1 h. The mixture was concentrated to obtain 5.3 g of white crystals with the following physical properties:

1HNMR (300 MHz, CD3OD) δ in ppm (two rotomers): 2.12 and 2.02 (heptuplet, 2H), 1.37 and 1.31 (s, 3H), 1.03 (ddd, 6H), 0.92 (dd, 6H)

13CNMR (75 MHz, CD3OD) 6 in ppm (two rotomers): 63.3, 59.3, 47.2, 33.8, 27.75, 24.5, 24.3, 24.2, 17.7, 17.4, 17.1, 16.9, 8.5

MS/EI: 263 (M^{+·}), 248, 220, 192, 152, 135, 107, 92

### EXAMPLE 3

### Preparation of 1,1-dimethylpropylamine

16 g of 1,1-diethylpropanol was treated in a way similar to that of example 2 to give the desired product with the following physical properties:

1HNMR (300 MHz, CDCl3) δ in ppm: 1.33 (qd, 6H), 0.82 (td, 9H)

13CNMR (75 MHz, CDCl3) δ in ppm: 53.2, 31.4, 7.6

MS/EI: 114 (M−1⁺), 98, 86, 69, 56

### EXAMPLE 4

### Preparation of N-1-methyl-1-isopropylisobutyl anisamide

0.10 g of 1-methyl-1-isopropylisobutylaminium chloride from example 2 and 0.20 g of pyridine were dissolved in 5 mL of MTBE and 0.16 g of p-anisoyl chloride were added. The mixture was stirred at room temperature overnight.

The resulting suspension was partitioned between MTBE and NaHCO₃ and extracted with MTBE. The organic layers were washed with brine, dried over MgSO₄ and concentrated to obtained 0.38 g of crude product, which was purified by recrystallization in hexane.

1HNMR (300 MHz, CDCl3) δ in ppm (two rotomers): 8.1 and 7.68 (d, 2H), 6.99 and 6.90 (d, 2H), 5.88 (broad s., 1H), 3.83 and 3.9 (s, 3H), 2.19 and 2.04 (heptuplet, 2H), 1.44 and 1.2 (s, 3H), 0.99 (dd, 6H), 0.86 (dd, 6H)

13CNMR (75 MHz, CDCl3) δ in ppm (two rotomers): 166, 163, 133, 128.5, 128, 114.5, 113.5, 57.9, 55.3, 49.7, 44.8, 27.0, 26.9, 25.5, 24.7, 24.4, 17.9, 8.5

### EXAMPLE 5

Following the same procedure according to Example 4 the compounds listed in Table 1 were synthesised.

TABLE 1

| No | Structure | Name | Physical Data |
|---|---|---|---|
| A | 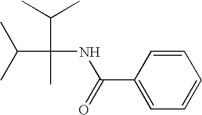 | N-(1-methyl-1-isopropylbutyl)-benzamide | MS: 233, 218, 190,122, 105, 77 |
| B | 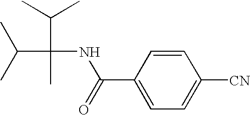 | N-(1-methyl-1-isopropylbutyl)-4-Cyano-benzamide | MS: 258, 243, 215,173, 130, 102 |
| C | 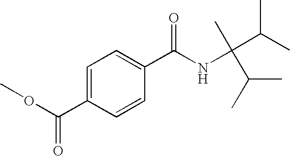 | N-(1-methyl-1-isopropyl-isobutyl) O-methylterephthalamate | MS: 291, 276, 248,163, 135 |
| D | 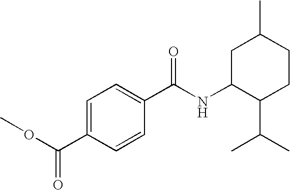 | N-(3-p-menthyl) O-methylterephthalamate | MS: 317, 302, 274,232, 180, 163, 135 |
| E | 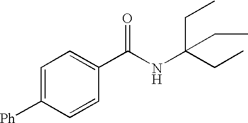 | N-(1,1-diethylpropyl)biphenyl-4-carboxamide | MS: 295, 266, 224,198, 181, 152 |
| F | 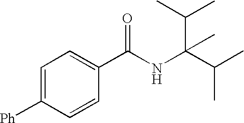 | N-(1-methyl-1-isopropyl-isobutyl) biphenyl-4-carboxamide | MS: 309, 294, 266,198, 181, 152 |
| G | 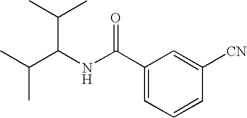 | N-(1-isopropylbutyl) 3-cyanobenzamide | MS: 244, 229, 201,130, 102 |
| H | 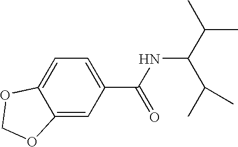 | N-(1-isopropyl-isobutyl)benzo[1,3]dioxole-5-carboxamide | MS: 263, 248, 220,165, 149, 121 |
| K | 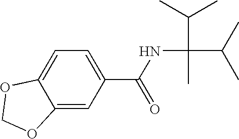 | N-(1-methyl-1-isopropyl-isobutyl)benzo[1,3]dioxole-5-carboxamide | MS: 277, 262, 234,166, 149, 121 |
| L | 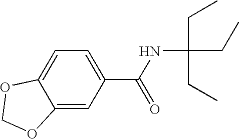 | N-(1,1-diethylpropyl)benzo[1,3]dioxole-5-carboxamide | MS: 263, 234, 165,149, 121 |
| M | 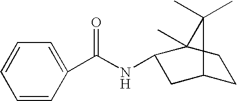 | N-bornyl benzamide | MS |

### EXAMPLE 6

### Cooling Effect

The cooling intensity of the compounds was determined by a trained panel of 4 to 8 people according to the isointensity method as described below.

Aqueous solutions of various concentrations of a chemical compound were prepared and tasted. The cooling intensity of each solution was compared to that of an aqueous solution of the reference compound at 2 ppm, namely menthol. The results are given in the list below.

| | | rel. cooling |
|---|---|---|
| Example | Chemical name | intensity |
| Ex. 4 | N-1-methyl-1-isopropylisobutyl | 1.1 |
| | anisamide | |
| Ex. 5A | N-(1-methyl-1-isopropylbutyl) | 0.1 |
| | benzamide | |
| Ex. 5B | N-(1-methyl-1-isopropylbutyl) | 1.0 |
| | 4-cyanobenzamide | |
| Ex. 5C | N-(1-methyl-1-isopropyl-isobutyl) | 1.7 |
| | O-methyl terephthalamate | |
| Ex. 5D | N-(3-p-menthyl) O-methyl | 2.5 |
| | terephthalamate | |
| Ex. 5E | N-(1,1-diethylpropyl) biphenyl- | 1.2 |
| | 4-carboxamide | |
| Ex. 5F | N-(1-methyl-1-isopropyl-isobutyl) | 1.5 |
| | biphenyl-4-carboxamide | |
| Ex. 5G | N-(1-isopropylisobutyl) | 0.2 |
| | 3-cyanobenzamide | |
| Ex. 5H | N-(1-isopropyl-isobutyl) | 0.9 |
| | benzo[1,3]dioxole-5-carboxamide | |
| Ex. 5K | N-(1-methyl-1-isopropyl-isobutyl) | 0.9 |
| | benzo[1,3]dioxole-5-carboxamide | |
| Ex. 5L | N-(1,1-diethylpropyl) | 0.3 |
| | benzo[1,3]dioxole-5-carboxamide | |
| Ex. 5M | N-bornyl benzamide | 0.6 |

### EXAMPLE 7

Application in mouthwash

| | | |
|---|---|---|
| Alcohol 95% | 177 | mL |
| Sorbitol 70% | 250 | g |
| Compound of example 4 as | 50 | mL |
| a 1% solution in alcohol | | |
| Peppermint oil, Terpeneless | 0.300 | g |
| Methyl salicylate | 0.640 | g |
| Eucalyptol | 0.922 | g |
| Thymol | 0.639 | g |
| Benzoic acid | 1.500 | g |
| Pluronic F127 | 5.000 | g |
| Sodium Saccharin | 0.600 | g |
| Sodium Citrate | 0.300 | g |
| Citric Acid | 0.100 | g |
| Water | q.s. 1 | liter |

All the ingredients were mixed. 30 mL of obtained solution was put in the mouth, swished around, gargled and spit out. A pleasant cooling sensation was felt in every area of the mouth.

### EXAMPLE 8

Application in toothpaste

| | |
|---|---|
| Opaque toothgel | 97.000 g |
| Compound of example 5B | 2.500 g |
| as a 2% solution in PG | |
| Peppermint oil, Terpeneless | 0.500 g |

The ingredients were mixed in the toothgel, a piece of toothgel was put on a toothbrush and a panelist's teeth were brushed. The mouth was rinsed with water and the water spit out. A longlasting cooling sensation was felt by the panelist in all areas of the mouth.

1. A method of providing a cooling sensation to the skin or the mucous membranes of the body by applying thereto a compound of Formula I 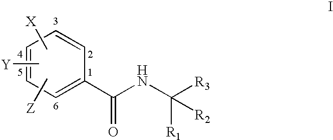
in which X is H or (CH₂)ₙ—R, n is 0 or 1, Y and Z are selected independently from the group consisting of H, OH, phenyl, C1-C4 straight or branched alkyl, and C₁-C₄ straight or branched alkoxy, or X and Y form together a bivalent radical selected from the group consisting of —O—CH₂—O—, —N═CH—O— and —N═CH—S— which forms together with the carbon atoms to which they are attached a 5-membered ring; and
R is a group with non-bonding electrons, R¹ is H or C₁-C₅ branched alkyl, R2 and R3 are C₁-C4 branched alkyls, or R² and R³ taken together form a monocyclic, bicyclic or tricyclic radical of up to 10 carbons provided that R¹, R² and R³ together comprise at least 6 carbons.

2. A method according to claim 1, in which R is selected from the group consisting of OH, OMe, NO₂, CN, Ac, SO₂NH₂, CHO, CO₂H, CONH₂, C₁-C₄ alkyl carboxylates, C₁-C₄ alkylamides and heterocycles.

3. A method according to claim 2, in which the heterocycle is selected from the group consisting of.

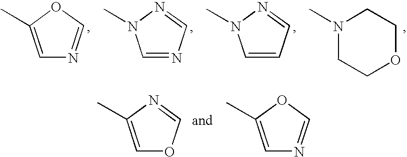

4. A method according to claim 1, in which R¹ is methyl and R² and R³ are isopropyl, or in which R¹, R² and R³ are all ethyl.

5. A method according to claim 1 in which R¹, R² and R³ are selected according to the following table:

| R¹ | R² | R³ |
|---|---|---|
| H | bornyl | |
| H | 3-p-menthyl | |
| H | isopropyl | isopropyl |
| methyl | isopropyl | isopropyl |
| ethyl | ethyl | ethyl |

6. A method according to claim 1, in which X is in the 4-position.

7. A method according to claim 6, in which Y and Z are independently selected from H, OH, OMe and methyl.

8. A method according to claim 1, in which the cooling effect is provided in a product that is to be orally ingested, applied to the skin or used in a tobacco product.

9. A composition that provides a cooling sensation to the skin or oral cavity, comprising an effective amount of a compound of Formula I 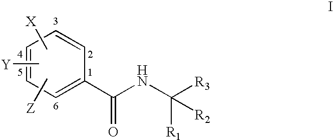
in which X is H or (CH₂)ₙ—R, n is 0 or 1, Y and Z are selected independently from the group consisting of H, OH, phenyl, C1-C4 straight or branched alkyl, and C₁-C₄ straight or branched alkoxy, or X and Y form together a bivalent radical selected from the group consisting of —O—CH₂—O—, —N═CH—O— and —N═CH—S— which forms together with the carbon atoms to which they are attached a 5-membered ring; and
R is a group with non-bonding electrons, R¹ is H or C₁-C₅ branched alkyl, R2 and R3 are C₁-C4 branched alkyls, or R² and R³ taken together form a monocyclic, bicyclic or tricyclic radical of up to 10 carbons provided that R¹, R² and R³ together comprise at least 6 carbons.

10. A compound of the formula I 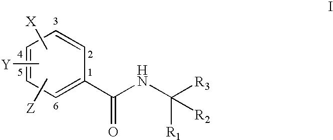
in which X is H or (CH₂)ₙ—R, n is 0 or 1, Y and Z are selected independently from the group consisting of H, OH, phenyl, C1-C4 straight or branched alkyl, and C₁-C₄ straight or branched alkoxy, or X and Y form together a bivalent radical selected from the group consisting of —O—CH₂—O—, —N═CH—O— and —N═CH—S— which forms together with the carbon atoms to which they are attached a 5-membered ring; and
R is a group with non-bonding electrons, R¹ is H or C₁-C₅ branched alkyl, R2 and R3 are C₁-C4 branched alkyls, or R² and R³ taken together form a monocyclic, bicyclic or tricyclic radical of up to 10 carbons provided that R¹, R² and R³ together comprise at least 6 carbons, and in which one of the following provisos applies:
(a) R¹ and X are not H, and, R¹, R², R³ and the carbon to which they are attached form an acyclic moiety;
(b) R¹ is H, and R², R³ and the carbon to which they are attached form an acyclic moiety, only one of R², R³ being isopropyl or tert-butyl;
(c) R¹ is H, R² and R³ are both isopropyl, and X is in the 4-position and is not H, halogen, Me, MeO, NO₂, aryl, methylenediaryl, N-(4-carbamimidoyl-phenyl)-6-methoxy-pyridine-2-carboxamide, N-(4-carbamimidoyl-phenyl)-benzamide, a heme derivative and R is not morpholine, N′-phenylpiperazine, phenylmercaptan, p-chlorophenylmercaptan, isoquinoline, an N-linked sulfonamide derivative or an aryl,
(d) R¹ is H and R² and R³ are both tert-butyl; X is not H;
(e) R¹ is H, R² and R³ together with the carbons to which they are attached form a p-menthane ring and X, Y and Z are not H;
(f) R¹ is H, R² and R³ together with the carbons to which they are attached form a p-menthane ring, Z is H and neither X or Y is H or OH;
g) R¹ is H, R² and R³ together with the carbons to which they are attached form a p-menthane ring, Z is H, Y is OH and X is neither formamide nor NO₂;
(h) R¹ is H, R² and R³ together with the carbons to which they are attached form a p-menthane ring, Z and Y are both H, and X is not H, COOH, quinolinylsulfonamide, CF₃, a methylenediaryl or a heme derivative.

11. A compound according to claim 10, in which the compound corresponds to one of Formulae II, III and IV, when provisos (a), (b) and (c) apply, and to Formula V when provisos (e), (f), (g) and (h) apply: [chemical structure]
